(19) United States
(12) Patent Application Publication
(10) Pub. No.: US 2012/0109304 A1
(43) Pub. Date: May 3, 2012

(54) MEDICAL IMPLANT AND METHOD FOR PHOTODYNAMIC THERAPY

(75) Inventors: Jonathan E. Balckwell, Arlington, TN (US); Keith E. Miller, Germantown, TN (US)

(73) Assignee: Warsaw Orthopedic, Inc., Warsaw, IN (US)

(21) Appl. No.: 12/915,787

(22) Filed: Oct. 29, 2010

(51) Int. Cl. A61F 2/44

(52) U.S. Cl. 623/17.12

(57) ABSTRACT

Embodiments of the invention include a medical implant for delivering photodynamic therapy. A device that includes a vertebral interbody device, alone or in combination with other complimentary elements, may be configured to deliver one or more therapeutic substances in combination with light emissions to provide photodynamic therapy.

### FIELD OF THE INVENTION

The present invention relates generally to the field of medical implants, and more particularly relates to a spinal implant that alone, or in combination with other components, is configured to activate a therapeutic substance by light emission.

### BACKGROUND

The use of therapeutic substances in combination with medical implants has beneficial characteristics in many treatments. Therapeutic substances may be useful in killing cancer cells, promoting healing, fighting infection and disease by killing various pathogens such as bacteria, viruses, and microorganisms, promoting favorable cellular activity, restricting unfavorable cellular activity, or any of a wide variety of beneficial results. At least one class of therapeutic substances, which may be referred to as a photosensitizer or photosensitizing agent, has an enhanced effect when exposed to light energy. Some photosensitizers produce a form of oxygen that kills nearby cells when the photosensitizer is exposed to particular wavelengths of light. Photosensitizers or agents that change in a patient's body to become a photosensitizer may be delivered intravenously and only activated where appropriate wavelengths of light are applied, or may be delivered more specifically to a site to be treated. It may be disadvantageous to deliver too much photosensitizer to a patient or to overexpose the photosensitizer to light. For example, the photosensitizer porfimer sodium may make a patient's eyes and skin sensitive to light for several weeks following treatment. Therefore, carefully controlling the amount of photosensitizer and light delivered to a patient may be advantageous.

It is a challenge in some treatments to deliver light of an effective wavelength and intensity to an effective location where an appropriate amount of photosensitizer has been delivered. It may also be a challenge in the art to provide medical implants that may be conveniently and securely placed in a patient to improve structural integrity of the patient's musculoskeletal system near sites that would benefit from treatment with a therapeutic substance. Such musculoskeletal treatments may include spinal procedures, such as vertebral augmentations or replacements, and spinal fusions. For example, it is sometimes necessary to remove one or more vertebrae, or a portion of the vertebrae, from the human spine in response to various pathologies. One or more of the vertebrae may become damaged as a result of tumor growth, or may become damaged by a traumatic or other event. Removal, or excision, of a vertebra may be referred to as a vertebrectomy. Excision of a generally anterior portion, or vertebral body, of the vertebra may be referred to as a corpectomy. An implant is usually placed between the remaining vertebrae to provide structural support for the spine as a part of a corpectomy or vertebrectomy. In some cases, the implant inserted between the vertebrae is designed to facilitate fusion between remaining vertebrae. In other cases, especially when treating tumors, the ultimate goal of the procedure is spinal stability, regardless of fusion. A successful procedure may decrease pain, preserve or enhance neurological function, and allow a patient greater mobility without an external orthosis. Sometimes an implant is designed to replace the function of the excised vertebra and discs. An implant may also be designed to only replace a spinal disc or part of a spinal disc. All or part of more than one vertebra may be damaged and require removal and replacement in some circumstances. If only a portion of a vertebral body and adjacent discs are removed and replaced, the procedure is called a hemi-vertebrectomy. Any of a spinal disc replacement or fusion, corpectomy, vertebrectomy, hemi-vertebrectomy or any other full or partial vertebral body excision may be referred to herein as a vertebral interbody procedure.

Improved devices may be configured to provide one or more of spinal stability, delivery of effective amounts of one or more therapeutic substances, and delivery of light, including light to activate a therapeutic substance.

### SUMMARY

An embodiment of the invention is a medical implant configured to treat tissue adjacent to the medical implant. The medical implant may include a vertebral interbody device having a first end, a substantially opposite second end, and one or more sides extending from the first end to the second end, wherein the one or more sides at least in part define an interior of the vertebral interbody device. The medical implant may also include at least one therapeutic substance reservoir, two or more nozzles in fluid communication with the at least one therapeutic substance reservoir, wherein the two or more nozzles are coupled to the vertebral interbody device to direct therapeutic substance from the interior of the vertebral interbody device, and an array of two or more light emission areas that are separately controllable to direct light away from the vertebral interbody device.

Another embodiment of the invention is a medical implant configured to treat tissue adjacent to the medical implant. The medical implant may include a vertebral interbody device having a first end, a substantially opposite second end, and one or more sides extending from the first end to the second end, wherein the one or more sides at least in part define an interior of the vertebral interbody device. The medical implant may also include an array of two or more light emission areas that are separately controllable to direct light away from the vertebral interbody device. The two or more light emission areas may be positioned substantially from a first point near the first end of the vertebral interbody device to a second point near the second end of the vertebral interbody device.

Yet another embodiment of the invention is a method of photodynamic therapy in the presence of a photosensitizer. The method may include implanting a medical implant comprising a vertebral interbody device having a first end, a substantially opposite second end, and one or more sides extending from the first end to the second end, wherein the one or more sides at least in part define an interior of the vertebral interbody device, and an array of two or more light emission areas that are separately controllable to direct light away from the vertebral interbody device. The method may also include activating one or more of the two or more light emission areas to apply light energy to tissue to which photosensitizer has been applied.

### BRIEF DESCRIPTION OF THE DRAWINGS

FIG. 1 is a perspective view on an embodiment of a medical implant configured to treat tissue adjacent to the medical implant.

FIG. 2 is a cross-sectional perspective view of implant illustrated in FIG. 1.

FIG. 3 is a top, plan view of an embodiment of a medical implant configured to treat tissue adjacent to the medical implant positioned next to a spinal cord.

### DETAILED DESCRIPTION

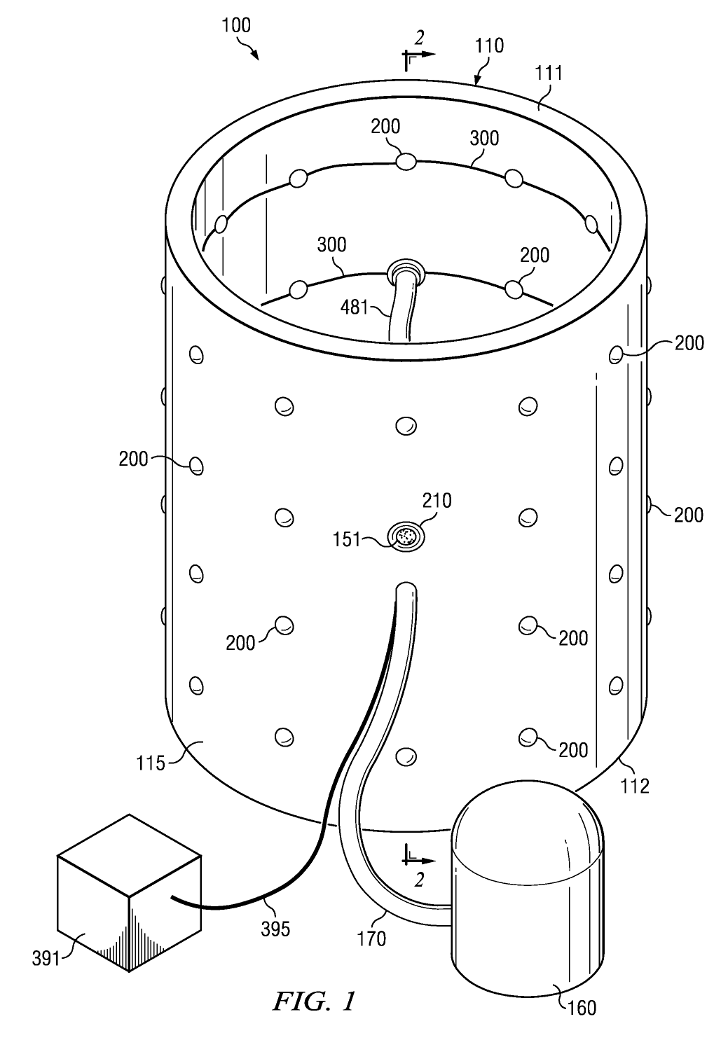

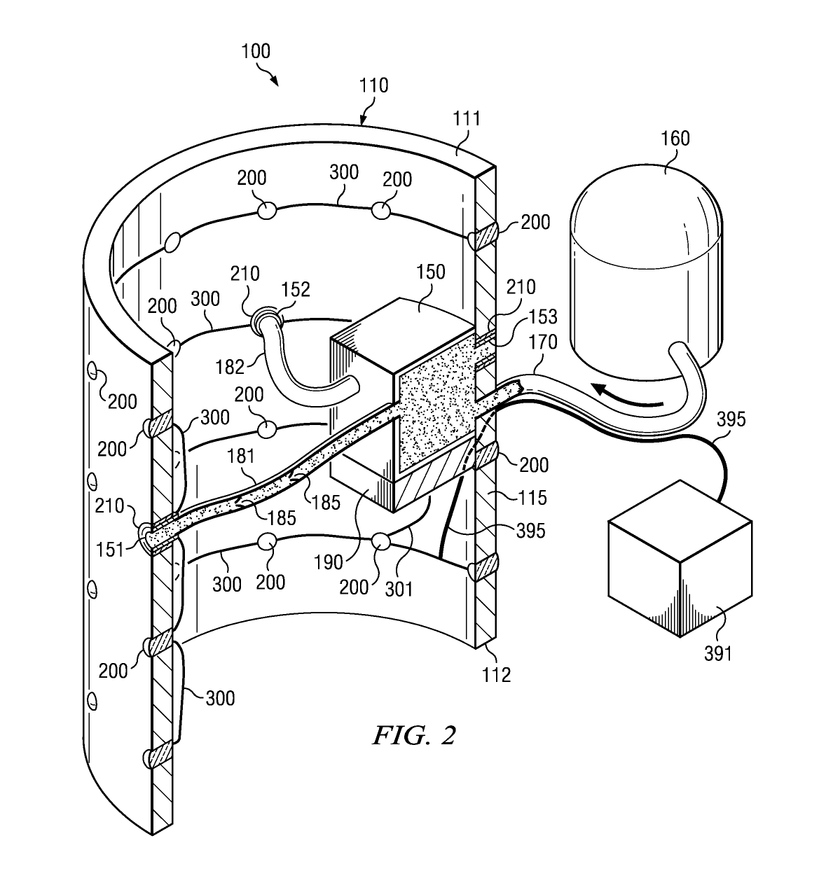

An embodiment of a medical implant 100 configured to treat tissue adjacent to the medical implant 100 is shown in FIGS. 1 and 2. As used herein, the term “adjacent” is not necessarily limited to a specific distance, but may refer to a distance to which a light emitted from the medical implant 100 will travel to or penetrate through tissue to be treated. The illustrated medical implant 100 includes a vertebral interbody device 110 having a first end 111 and a substantially opposite second end 112. A side 115 of the vertebral interbody device 110 extends from the first end 111 to the second end 112. The side 115 defines an interior of the vertebral interbody device 110. The interior is said to be defined by the side 115 in that the side 115 approximately lays out a boundary of the interior of the vertebral interbody device 110. In other embodiments, two or more sides may be joined together in the formation of a vertebral interbody device.

The illustrated vertebral interbody device 110 is a vertebral body replacement device. In other embodiments, a vertebral interbody device may be a multilevel vertebral body replacement device, an implant approximating the size of a single disc between vertebral bodies, a multilevel device place for any purpose, or a device that replaces any portion of a vertebral structure, including but not limited to, bone, disc material, ligaments, and connective tissue of any type.

In other embodiments, a vertebral interbody device may include a mesh material or may include holes of any shape, slots, or may not include openings. The vertebral interbody device may be constructed in whole or in part of any biocompatible material, including but not limited to, titanium, polyetheretherketone (PEEK), cobalt chrome, stainless steel, or any biocompatible metal, metal alloy, polymer, or a bone or bone-based material such as allograft, xenograft, demineralized bone, or autograft. The illustrated vertebral interbody device 110 is a generally round cross-sectional shape. In other embodiments, a vertebral interbody device may have a lateral periphery of one or more walls having a cross-sectional shape that is substantially oval, kidney shape, triangle, rectangle, square, any polygonal or curved shape, or any combination of shapes.

The medical implant 100 shown includes a therapeutic substance reservoir 150 located within an interior of the vertebral interbody device 110. In other embodiments, a therapeutic substance reservoir is located outside of the interior of a vertebral interbody device. For example and without limitation, a therapeutic substance reservoir may be located outside of the interior of a vertebral interbody device, but within a patient's body. In other embodiments, a therapeutic substance reservoir may be located outside of the interior of a vertebral interbody device, and outside of a patient's body. As shown in FIGS. 1 and 2, there is a second therapeutic substance reservoir 160. The second therapeutic substance reservoir 160 may be located inside or outside of a patient's body. The second therapeutic substance reservoir 160 in the illustrated embodiment may serve as a supply or re-supply mechanism for the therapeutic substance reservoir 150 via a conduit 170. More than one therapeutic substance reservoir may be employed with some embodiments of the medical implant. The more than one therapeutic substance reservoir may be located together or separately at any effective location.

The medical implant 100 also includes nozzles 151, 152, 153 in fluid communication with the therapeutic substance reservoir 150. Any number of nozzles may be present with other embodiments. The nozzles 151, 152, 153 illustrated are coupled to the vertebral interbody device 110 to direct therapeutic substance from the interior or the vertebral interbody device 110. Nozzles may be located anywhere along a medical implant to direct therapeutic substance in a clinically advantageous way. The nozzles 151, 152 are shown in FIG. 6 in fluid communication with the therapeutic substance reservoir 150 through respective tubes 181, 182. The nozzle 153 is illustrated directly connected to the therapeutic substance reservoir 150 to provide fluid communication. Other embodiments may include any number or type of fluid communication mechanisms. The nozzles 151, 152, 153 are directed from the interior of the vertebral interbody device 110 substantially transversely to the side 115. In other embodiments, nozzles may be directed from the interior in any clinically effective direction.

The medical implant 100 may also include one or more valves to control flow of therapeutic substance from the nozzles 151, 152, 153. Valves may be located at a nozzle, along any of the respective tubes, at the therapeutic substance reservoir, outside of the medical implant, or at any other effective location. Valves may be controlled by any effective signal mechanism, including but not limited to, electrical, radio frequency, and pressure. Valves may be signaled directly or through one or more controller devices. By way of non-limiting example in FIG. 2, one or more signal wires may be integrated with the conduit 170 to carry signals into the vertebral interbody device 110. The signal wires may be connected to a controller 190. The controller 190 may be configured to generate an increased pressure within the therapeutic substance reservoir 150, or elsewhere in the device, or to open and close valves within the therapeutic substance reservoir 150, at the nozzles 151, 152, 153, and along tubes 181, 182. Embodiments of the tubes and nozzles may also include passive, one-way valves to control the direction of flow therapeutic substance. Example one-way valves 185 are shown in the tube 181. Any effective combination of controlled and passive valves is contemplated. Pressure or flow within embodiments of a therapeutic substance reservoir, nozzles, and tubes may be generated by any effective mechanism. Example mechanisms include but are not limited to, pistons, bellows, piezoelectric driven pumps, and bubble jet spray mechanisms similar to mechanisms used with printing devices. By way of non-limiting example, nozzles may be directed towards a region of a spine or surrounding area from which a tumor has been removed or where a recurrence of a tumor has been detected.

In some embodiments, two or more therapeutic substances may be directed through different nozzles of the medical implant. Two or more therapeutic substances may be directed through the same nozzle at different times or as a mixture. The medical implant 100 illustrated in FIG. 2, and other similar devices, may include any of the therapeutic substances disclosed herein in any composition, form, or mixture.

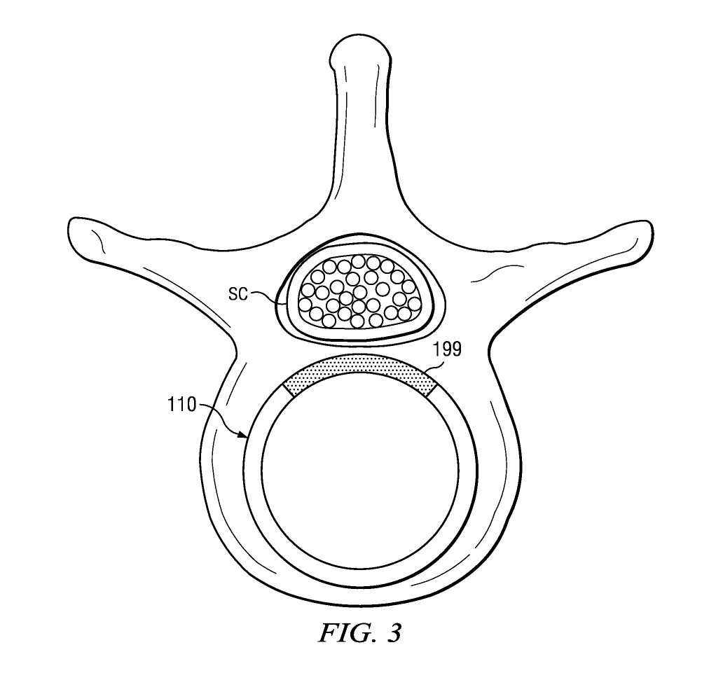

In some embodiments, no nozzles are directed from the interior of a vertebral interbody device through certain, designated portions of the vertebral interbody device. In these embodiments, the designated portions may be configured to be implanted adjacent to a spinal cord. A non-limiting example of this configuration is the vertebral interbody device 110 illustrated in FIG. 3 with a designated portion 199 through which no nozzles are directed. This configuration may be useful to avoid directing therapeutic substances toward a spinal cord (SC) and other major neural structures. Similarly, in some embodiments such a configuration may be used to avoid directing therapeutic substances toward vascular structures, such as, for example, the great vessels along an anterior portion of a spinal column. Any other anatomical part, including anatomical parts sensitive to a therapeutic, may be effectively avoided by selective direction of a therapeutic substance. Non-therapeutic regions or regions containing particular therapeutic substances may be placed next to any area helpful in achieving a desired clinical result, including but not limited to, anterior, posterior, oblique, lateral, medial, caudal, and cephalad.

A therapeutic substance may comprise one or more of the following: photosensitizer, photosensitizer precursor, antibiotics, antiseptics, analgesics, bone growth promoting substances, anti-inflammatants, anti-coagulants, antifungal agents, steroids, enzymes, immunosuppressants, antithrombogenic compositions, vaccines, hormones, growth inhibitors, growth stimulators, chemotherapy drugs, and the like. A therapeutic substance may be any drug or bioactive agent which can serve a useful therapeutic or even diagnostic function when released into a patient. More than one therapeutic substance may be employed with various embodiments of the invention.

A therapeutic substance may include pharmaceuticals that target particular cells, such as but not limited to, cancer cells. A therapeutic substance may be any radiopharmaceutical or radionuclide, for example. A therapeutic substance may be a pure bone-seeking radioisotope, such as strontium-89 and phosphorus-32 or a radioisotope that has been combined with other bone-seeking agents, such as samarium-153, rhenium-186, and iodine-131.

A therapeutic substance may include a photosensitizer, photosensitizing agent, or photosensitizer precursor. A photosensitizer of various embodiments may produce a form of oxygen that kills nearby cells when the photosensitizer is exposed to particular wavelengths of light. For example, the photosensitizer porfimer sodium, which may be sold under the brand name, PHOTOFRIN, may be injected into tissue, and when the tissue is exposed to light, such as laser light, an excited singlet oxygen may be created, which is capable of killing nearby cells. Other photosensitising agents may include, without limitation, tetracyclines, sulphonamides, phenothiazines, sulphonylureas, thiazide diuretics, and griseofulvin. Photosensitizer precursors are substances that are capable of further reacting or converting in a patient's body to produce a photosensitizer. Example photosensitizes precursors include, but are not limited to, aminolevulinic acid, methyl aminolevulinate, and levulinic acid. Additional commercially available and developmental photosensitizers and photosensitizer precursors include, but are not limited to, AMPHINEX, ANTRIN, BF-200 ALA, FOSCAN, HEXVIX, LASERPHYRIN, LEVULAN, METVIX, PHOTOCHLOR, PHOTOSENS, PHOTREX, and VISUDYNE.

A therapeutic substance may include a DNA-damaging agent, such as chlorambucil, cyclophosphamide or melphalan, collectively referred to as alkylating agents. These DNA-damaging agents damage the DNA so severely that the cancer cell is killed. Other DNA-damaging agents, such as carboplatinum, attach to the DNA and prevent the cancer cell from growing.

A therapeutic substance may include antitumor antibiotics, such as daunorubicin, doxorubicin, idarubicin, and mitoxantrone, which insert themselves into the DNA of a cancer cell, prevent the DNA from functioning normally, and often kill the cancer cell.

A therapeutic substance may include antimetabolites, such as methotrexate, fludarabine, and cytarabine. These drugs mimic substances that the cancer cell needs to build DNA and RNA. When a cancer cell uses the antimetabolite instead of the natural substances, it cannot produce normal DNA or RNA, and the cell dies.

A therapeutic substance may include DNA-repair enzyme inhibitors, such as etoposide or topotecan. These inhibitors attack the cancer cell proteins that normally repair any damage to the cell DNA. Repair of DNA damage is a normal and vital process in the cell, without which the cancer cell is much more susceptible to damage and is prevented from growing.

A therapeutic substance may include vincristine or vinblastine. These agents damage cancer cells by blocking mitosis. This prevents the cancer cells from dividing and multiplying.

A therapeutic substance may include antibodies that are made specifically to attach to cancer cells. Once these antibodies attach to the cancer cells, the antibodies interfere with the cells' functions and kill the cells. Some antibodies may also be linked to a toxin or radioactive substance. When these antibodies attach to cancer cells, one or more of the antibodies, the toxin, and the radioactive substance work to kill cancer cells.

A therapeutic substance may include radiation sources that remain physically within a device, but that emit radiation in a desired direction from the device. By way of non-limiting example, a radiation source may be a radionuclide, such as I-125, I-131, Yb-169, Ir-192 or other radionuclides that emit photons, beta particles, gamma radiation, or other therapeutic energy or substances. A radioactive therapeutic substance may also be a fluid made from any solution of radionuclide(s), e.g., a solution of I-125 or I-131, or a radioactive mixture may be produced using a slurry of a suitable fluid containing small particles of solid radionuclides, such as Au-198, Y-90. Radionuclides may also be provided in a gel. One radioactive material useful in some embodiments is Iotrex®, a nontoxic, water soluble, nonpyrogenic solution containing sodium 3-(125I)iodo-4-hydroxybenzenesulfonate (125I-HBS), available from Proxima Therapeutics, Inc. of Alpharetta, Ga. Radioactive micro spheres of the type available from the 3M Company of St. Paul, Minn., may also be incorporated into or introduced into a device. A radioactive source may be preloaded into a medical implant at the time of manufacture or loaded after the medical implant has been implanted.

A therapeutic substance may include any antibiotic suitable for use in a human. As used herein, “antibiotic” means an antibacterial agent. The antibacterial agent may have bacteriostatic and/or bacteriocidal activities. Nonlimiting examples of classes of antibiotics that may be used include tetracyclines (e.g. minocycline), rifamycins (e.g. rifampin), macrolides (e.g. erythromycin), penicillins (e.g. nafcillin), cephalosporins (e.g. cefazolin), other beta-lactam antibiotics (e.g. imipenem, aztreonam), aminoglycosides (e.g. gentamicin), chloramphenicol, sulfonamides (e.g. sulfamethoxazole), glycopeptides (e.g. vancomycin), quinolones (e.g. ciprofloxacin), fusidic acid, trimethoprim, metronidazole, clindamycin, mupirocin, polyenes (e.g. amphotericin B), azoles (e.g. fluconazole) and beta-lactam inhibitors (e.g. sulbactam). Nonlimiting examples of specific antibiotics that may be used include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin. Other antibiotics may also be used.

To enhance the likelihood that bacteria will be killed or inhibited, it may be desirable to combine one or more antibiotics. It may also be desirable to combine one or more antibiotics with one or more antiseptics. Agents having different mechanisms of action and/or different spectrums of action may be most effective in achieving such an effect.

Any antiseptic suitable for use in a human may be used as or as part of a therapeutic substance. As used herein, “antiseptic” means an agent capable of killing or inhibiting the growth of one or more of bacteria, fungi, or viruses. Antiseptic includes disinfectants. Nonlimiting examples of antiseptics include hexachlorophene, cationic bisiguanides (i.e. chlorhexidine, cyclohexidine) iodine and iodophores (i.e. povidone-iodine), para-chloro-meta-xylenol, triclosan, furan medical preparations (i.e. nitrofurantoin, nitrofurazone), methenamine, aldehydes (glutaraldehyde, formaldehyde), silver sulfadiazine and alcohols. It may be desirable that the one or more antiseptics selected kill or inhibit the growth of one or more microbes that are associated with infection following surgical implantation of a medical device. Such bacteria may include Stapholcoccus aureus, Staphlococcus epidermis, Pseudomonus auruginosa, and Candidia. To enhance the likelihood that microbes will be killed or inhibited, it may be desirable to combine one or more antiseptics. It may also be desirable to combine one or more antiseptics with one or more antibiotics. Antimicrobial agents having different mechanisms of action and/or different spectrums of action may be most effective in achieving such an effect.

A therapeutic substance may comprise an antimicrobial material including metals known to have antimicrobial properties, such as silver, gold, platinum, palladium, iridium, tin, copper, antimony, bismuth, selenium and zinc. Compounds of these metals, alloys containing one or more of these metals, or salts of these metals may be coated onto the surface of a device or added to the material from which a device is made during the manufacture of the device or compounded into the base material. One therapeutic substance will contain silver ions and may be obtained through the use of silver salts, such as silver acetate, silver benzoate, silver carbonate, silver iodate, silver iodide, silver lactate, silver laurate, silver nitrate, silver oxide, silver palmitate, silver protein, or silver sulfadiazine, among others. In an embodiment where selenium is used, the selenium may be bonded to the surface of a device, providing an antimicrobial coating.

A therapeutic substance may also comprise an osteoconductive, osteogenic, or osteoinductive material. For example and without limitation, a therapeutic substance may include various bioceramic materials, calcium phosphate and other members of the calcium phosphate family, fluorapatite, bioactive glass, and collagen-based materials. Members of the calcium phosphate family include materials such as hydroxyapatite, α-tricalcium phosphate, β-tricalcium phosphate, tetracalcium phosphate, dicalcium phosphate dihydrate, ocatacalcium phosphate, and the like. A therapeutic substance may include an osteoinductive or osteogenic materials such as osteoblast cells, platelet-derived growth factors (PDGFs), bone morphogenetic proteins (BMPs), insulin-like growth factors (IGFs), basic fibroblast growth factor (bFGF), cartilage derived morphogenetic protein (CDMP), growth and differentiation factors (GDFs), LIM mineralization proteins, transforming growth factor beta family (TGF-β), and other bone proteins, such as CD-RAP. These proteins can be recombinantly produced or obtained and purified from an animal that makes the proteins without the use of recombinant DNA technology. Recombinant human BMP is referred to as “rhBMP”; recombinant human GDF is referred to as “rhGDF”. Any bone morphogenetic protein is contemplated, including bone morphogenetic proteins designated as BMP-1 through BMP-18. Mimetics of growth factors can also be used in the devices of the present invention for inducing the growth of bone.

Each BMP may be homodimeric, or may be heterodimeric with other BMPs (e.g., a heterodimer composed of one monomer each of BMP-2 and BMP-6) or with other members of the TGF-β superfamily, such as activins, inhibins and TGF-β1 (e.g., a heterodimer composed of one monomer each of a BMP and a related member of the TGF-β superfamily). Any of these substances may be used individually or in mixtures of two or more. One or more statins may also be included in a therapeutic substance. Non-limiting examples of statins that may be included in the devices of the present invention include atorvastatin, cerivastatin, fluvastatin, lovastatin, mavastatin, pitavastatin, pravastatin, rosuvastatin and simvastatin. The therapeutic substance may include various other organic species known to induce bone formation, and combinations thereof.

Any of these therapeutic substances may be delivered through the therapeutic substance reservoir, by direct application, or through a systemic distribution such as an intravenous injection. Any combination of such uses may be employed. For example, photosensitizer may be applied from the therapeutic substance reservoir in the presence of another therapeutic substance and light applied to the tissue to be treated. By way of further example, photosensitizer may be applied systemically, another therapeutic substance applied from the therapeutic substance reservoir, and light applied to the tissue to be treated. Any other effective combination of therapeutic substance uses is contemplated herein. Any mixture of therapeutic substances may be applied, and any number of separate therapeutic substance reservoirs may be used in various embodiments.

Embodiments of a medical device configured to treat tissue adjacent to the medical device may include an array of two or more light emission areas to direct light away from the medical device. For example and without limitation, the medical device 100 illustrated in FIGS. 1 and 2 includes an array of lights 200, 210 that are separately controllable. The array of lights 200, 210 is configured to direct light away from the vertebral interbody device 110. The array of lights 200, 210 may include individual light generating or emitting devices at each location along the array or may include a lesser number of light generating or emitting devices that are directed to more than one point of the array by waveguides or other light conveying mechanisms. Each of the light emission areas may be separately controlled to provide for application of light in particular locations and for particular amounts of time. Groups of light emission areas may be controlled together.

Light for an array of lights may be generated by any form of light source. For example and without limitation, light may come from a laser, a light emitting diode (LED), an incandescent bulb, or a florescent bulb or apparatus, or any combination of such sources. Light for the array of lights 200, 210 illustrated may be generated by any form of light. As illustrated, each light 200, 210 is a light emission area that generates light in response to an electrical signal applied over a network of electrical supply wires 300. In the illustrated embodiment, internal supply lead 301 (FIG. 2) is routed from the controller 190 to the network of electrical supply wires 300. In other embodiments, a separate light controller 391 that is distinct from the controller 190 may be provided to signal operation of the lights. Either or both of the controller 190 and the light controller 391 may be configured to selectively turn on and off one or more of the light emission areas. Alternatively or in addition, the light emission areas may be activated at various levels of intensity. Light emission areas may be turned off and on at various levels of intensity at scheduled times to delivery a predetermined treatment plan. Light emission may be further coordinated with the delivery of therapeutic substances in the performance of a treatment plan. In some embodiments, control of light emission is electrically integrated with a valve controller, as for example, in the controller 190.

An external supply lead 395 is illustrated in FIG. 2 electrically connecting from the light controller 391 to the electrical supply wires 300. The supply lead 301 may include multiple wires for separately illuminating various of the lights 200, 210. Separate, grouped, or unitary control may also be enacted by signals sent to the lights 200, 210 over the electrical supply wires 300, by wireless signal, or by any other effective signal or mechanism. Lead or other control wires, such as the external supply lead 395, may be a part of the conduit 170 or may be separate from the conduit 170. In some embodiments, a control wire, such as the external supply lead 395, may be routed through the circuitry of one or more of the device controllers, such as the controller 190, rather than connected with the electrical supply wires 300, as illustrated in FIG. 2.

The light emission areas embodied in the lights 210 show light emission areas integrated with nozzles 151, 152, 153 of the medical implant. As shown, the lights 210 form a concentric ring around each of the respective nozzles 151, 152, 153. This configuration allows for light to be transmitted directly in the area closest to the supply of therapeutic substance. In other embodiments, a different proportion, including possibly all of the light emission areas, may include an integrated nozzle. Light emission areas may be distributed at any point along a vertebral interbody device. As shown in FIGS. 1 and 2, lights 200, 210 are distributed around the full periphery of the vertebral interbody device 110, substantially from the first end 111 to the second end 112 of the vertebral interbody device 110. The light emission areas embodied in the lights 200, 210 are shown positioned substantially from a first point near the first end 111 to a second point near the second end 112, and are positioned at two or more radial locations relative to an axis of the vertebral interbody device 110 that passes through the first and second ends 111, 112.

In an embodiment where light is distributed to different points along an array from a central source or sources, the elements designated as electrical supply wires 300 may be waveguides or optical cables of any effective type. Control of transmitted light to various points on the array may be accomplished by any effective means known in the art.

An embodiment is illustrated in FIG. 3 where the designated portion 199 of the interbody device, which is described above as having no nozzles directed through the designated portion 199, may also not have any light emission areas or may have all of the light emission areas turned off for a course of treatment. This configuration may be useful to avoid directing light and associated therapy toward a spinal cord (SC) and other major neural structures. Similarly, in some embodiments such a configuration may be used to avoid directing light and subsequent therapy toward vascular structures, such as, for example, the great vessels along an anterior portion of a spinal column. Any other anatomical part, including anatomical parts sensitive to a treatment, may be effectively avoided by selective direction of light from a device. Greater or fewer numbers of light emission areas may be located at any point on a vertebral interbody device. The number may be increased generally where greater light intensity is required, or light emission areas may be placed in areas where treatment is more likely to be needed in various parts of the anatomy where a device is planned for use.

The full disclosure, including devices, structures, and methods, of U.S. application Ser. No. 12/847,069, entitled “Vertebral Body Replacement Device Configured to Deliver a Therapeutic Substance” and U.S. Pat. App. Pub. No. 2005/0175658, entitled, “Implant Having a Photocatalytic Unit” are hereby incorporated by reference herein in their entirety.

An embodiment of the invention is a method of photodynamic therapy in the presence of a photosensitizer. This method embodiment may include implanting a medical implant and activating one or more light emission areas to apply light energy to tissue to which photosensitizer has been applied.

The medical implant used may include any of the variations of the vertebral interbody device described herein, such as vertebral interbody device 110, as well as other devices capable of use with the acts of the method described. A suitable vertebral interbody device may have a first end, a substantially opposite second end, and one or more sides extending from the first end to the second end. The one or more sides at least in part define an interior of the vertebral interbody device. The medical implant may also include an array of two or more light emission areas that are separately controllable to direct light away from the vertebral interbody device. Variations of the medical device 100 described above are also applicable to the device used as part of the described method.

Another act of some method embodiments is activating one or more of the two or more light emission areas to apply light energy to tissue to which photosensitizer or photosensitizer precursor has been applied. Photosensitizer or photosensitizer precursor may be applied systemically to a patient by injection, intravenous drip, etc. Alternatively, photosensitizer or photosensitizer precursor may be applied more directly to a treatment site. For example, photosensitizer may be applied to a treatment site directly through a vertebral interbody device with a therapeutic substance reservoir. Some photosensitizers and photosensitizer precursors require a period of waiting prior to application of light for effective photodynamic therapy. For example and without limitation, a period of 48 hours between application of photosensitizer or photosensitizer precursor may be prescribed with some substances. In some embodiments, photosensitizer or photosensitizer precursor is applied after implanting the medical implant. In some embodiments, photosensitizer or photosensitizer precursor is applied before implanting the medical implant.

Treatment of specific tissue may be controlled by the directed application of light from the medical device. For example and without limitation, a light emission area may be placed adjacent to a tumor or an area from which most of a tumor was believed to be removed. Later activation of this light emission area will focus treatment on such a site. In some embodiments, treatment is restricted from application near sensitive areas such as a spinal cord or nerve root by ensuring that no light is applied in the direction of a spinal cord or nerve root. In some embodiments, specific amounts of light may be applied for particular periods of time in a manner that is known or discovered to provide treatment without damage to sensitive or critical tissues.

Some embodiments include cycling two or more light emission areas through various intensities for various amounts of time to achieve therapeutic goals. The two or more light emission areas may be cycled between on and off states or may be cycled through intensities intermediate of fully on and off states in addition to fully on and off states. In some embodiments, the effectiveness of an ongoing therapy routine may be evaluated during a treatment cycle. In response to such an evaluation, the therapy applied may be adjusted by altering the state of at least one of the two or more light emission areas. In some embodiments, additional photosensitizer or photosensitizer precursor may be applied during a treatment cycle to achieve a clinical objective. This additional photosensitizer or photosensitizer precursor may be applied systemically, through, or from within the medical implant. In addition, waiting periods absent of application of therapeutic substance or light emission may be interjected during a treatment cycle to create a successful treatment regimen.

Terms such as adjacent, around, near, opposite, side and the like have been used herein to note relative positions. However, such terms are not limited to specific coordinate orientations, but are used to describe relative positions referencing particular embodiments. Such terms are not generally limiting to the scope of the claims made herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. A medical implant configured to treat tissue adjacent to the medical implant comprising:
a vertebral interbody device having a first end, a substantially opposite second end, and one or more sides extending from the first end to the second end, wherein the one or more sides at least in part define an interior of the vertebral interbody device;
at least one therapeutic substance reservoir;
two or more nozzles in fluid communication with the at least one therapeutic substance reservoir, wherein the two or more nozzles are coupled to the vertebral interbody device to direct therapeutic substance from the interior of the vertebral interbody device;
an array of two or more light emission areas that are separately controllable to direct light away from the vertebral interbody device.

2. The medical implant of claim 1 wherein the therapeutic substance reservoir is within the interior of the vertebral interbody device.

3. The medical implant of claim 1 wherein the therapeutic substance reservoir is outside of the interior of the vertebral interbody device.

4. The medical implant of claim 1, further comprising one or more valves to regulate flow of therapeutic substance from the two or more nozzles.

5. The medical implant of claim 4, further comprising a controller that operates the one or more valves.

6. The medical implant of claim 1 wherein at least one of the two or more light emission areas is integrated with at least one of the two or more nozzles.

7. The medical implant of claim 1 wherein the array of two or more light emission areas extends substantially from the first end of the vertebral interbody device to the second end of the vertebral interbody device.

8. The medical implant of claim 1, further comprising a light controller configured to selectively turn on and off one or more of the light emission areas.

9. The medical implant of claim 8 wherein the light controller is electrically integrated with a valve controller that regulates flow of the therapeutic substance.

10. The medical implant of claim 1, further comprising a therapeutic substance containing one or more of the following substances: photosensitizer, photosensitizer precursor, antibiotic, antiseptic, analgesic, anti-inflammatant, anti-coagulant, antifungal, steroid, enzyme, immunosuppressant, antithrombogenic composition, vaccine, hormone, growth inhibitor, growth stimulator, chemotherapy drugs, biodegradable non-therapeutic, and bone growth promoting.

11. A medical implant configured to treat tissue adjacent to the medical implant comprising:
a vertebral interbody device having a first end, a substantially opposite second end, and one or more sides extending from the first end to the second end, wherein the one or more sides at least in part define an interior of the vertebral interbody device; and
an array of two or more light emission areas that are separately controllable to direct light away from the vertebral interbody device;
wherein the two or more light emission areas are positioned substantially from a first point near the first end of the vertebral interbody device to a second point near the second end of the vertebral interbody device.

12. The medical implant of claim 11 wherein the two or more light emission areas are both positioned substantially from a first point near the first end of the vertebral interbody device to a second point near the second end of the vertebral interbody device, and are positioned at two or more radial locations relative to an axis of the vertebral interbody device that passes through the first and second ends of the vertebral interbody device.

13. The medical implant of claim 11, further comprising a light controller configured to selectively turn on and off one or more of the light emission areas.

14. A method of photodynamic therapy in the presence of a photosensitizer comprising:
implanting a medical implant comprising:
a vertebral interbody device having a first end, a substantially opposite second end, and one or more sides extending from the first end to the second end, wherein the one or more sides at least in part define an interior of the vertebral interbody device, and
an array of two or more light emission areas that are separately controllable to direct light away from the vertebral interbody device;
activating one or more of the two or more light emission areas to apply light energy to tissue to which photosensitizer or photosensitizer precursor has been applied.

15. The method of claim 14, further comprising applying photosensitizer or photosensitizer precursor after implanting the medical implant.

16. The method of claim 14 wherein no light is applied in the direction of a spinal cord or nerve root.

17. The method of claim 14, further comprising cycling at least one of the two or more light emission areas between two or more states.

18. The method of claim 14, further comprising evaluating the effectiveness of the therapy applied and altering the state of at least one of the two or more light emission areas in response.

19. The method of claim 14, further comprising applying additional photosensitizer or photosensitizer precursor.

20. The method of claim 19 wherein the additional photosensitizer or photosensitizer precursor is applied from within the medical implant.
